(19) United States
(12) Patent Application Publication
(10) Pub. No.: US 2022/0225889 A1
(43) Pub. Date: Jul. 21, 2022

(54) MAGNETIC RESONANCE IMAGING APPARATUS

(71) Applicant: CANON MEDICAL SYSTEMS CORPORATION, Tochigi (JP)

(72) Inventor: Naotaka SAKASHITA, Utsunomiya (JP)

(73) Assignee: CANON MEDICAL SYSTEMS CORPORATION, Tochigi (JP)

(21) Appl. No.: 17/645,109

(22) Filed: Dec. 20, 2021

(30) Foreign Application Priority Data
Jan. 20, 2021 (JP) 2021-007120

(51) Int. Cl. A61B 5/055; A61B 5/026; A61B 5/00

(52) U.S. Cl. CPC A61B 5/055; A61B 5/0042; A61B 5/0263

(57) ABSTRACT

A magnetic resonance imaging apparatus according to an embodiment includes processing circuitry. The processing circuitry sets an imaging region and a labeling region based on at least a blood vessel structure, the imaging region being a region of a matrix of a plurality of divided voxels including different blood vessel regions, respectively, the labeling region being a region to which a labeling pulse for labeling blood flowing into the imaging region is applied. The processing circuitry acquires data of the imaging region by applying the labeling pulse to the labeling region by using an arterial spin labeling (ASL) method. The processing circuitry generates an image based on the data. The processing circuitry determines anomaly of the blood vessel regions by comparing signal values of the voxels included in the image.

### CROSS-REFERENCE TO RELATED APPLICATIONS

This application is based upon and claims the benefit of priority from Japanese Patent Application No. 2021-007120, filed on Jan. 20, 2021; the entire contents of which are incorporated herein by reference.

### FIELD

Embodiments described herein relate generally to a magnetic resonance imaging apparatus.

### BACKGROUND

Conventionally, as an example of magnetic resonance imaging (MRI) apparatuses, an MRI apparatus using a static magnetic field having a magnetic field intensity lower than the magnetic field intensity of a typical MRI apparatus that is installed at a hospital or the like. Such an MRI apparatus of a low magnetic field intensity normally can operate with a commercial power source and has a compact apparatus size, and thus is called a portable MRI.

Recently, it has been discussed to mount such a portable MRI on an ambulance and use the portable MRI for solutions in the ambulance. For example, in an examination of cerebral infarction, it is important to specify the existence of a large vessel occlusion (LVO) in the anterior circulation system at an early point after the start of symptoms. Thus, it has been discussed to determine LVO existence in an ambulance by using a portable MRI.

However, a portable MRI, which typically has a low magnetic field intensity, has constraints such as a low time-of-flight (TOF) effect, a long image capturing time, a low resolution, and a low signal-to-noise ratio (SNR). Thus, with a portable MRI, it is difficult to capture an image of a blood vessel by using a typical TOF method as a method of MR angiography (MRA), and accordingly, it is difficult to determine LVO existence.

### BRIEF DESCRIPTION OF THE DRAWINGS

FIG. 1 is a diagram illustrating an exemplary configuration of an MRI apparatus according to a first embodiment;

FIG. 2 is a flowchart illustrating the procedure of processing performed by the MRI apparatus according to the first embodiment;

FIG. 3 is a diagram illustrating exemplary detection of the boundary between an LV and a non-LV, which is performed by a setting function according to the first embodiment;

FIG. 4 is a diagram illustrating exemplary detection of the boundary between the LV and the non-LV, which is performed by the setting function according to the first embodiment;

FIG. 5 is a diagram illustrating exemplary detection of the boundary between the LV and the non-LV, which is performed by the setting function according to the first embodiment;

FIGS. 6A and 6B are diagrams illustrating exemplary detection of the boundary between the LV and the non-LV, which is performed by the setting function according to the first embodiment;

FIG. 7 is a diagram illustrating exemplary labeling slab setting performed by the setting function according to the first embodiment;

FIG. 8 is a diagram illustrating exemplary labeling slab setting performed by the setting function according to the first embodiment;

FIG. 9 is a diagram illustrating exemplary labeling slab setting performed by the setting function according to the first embodiment;

FIGS. 10A and 10B are diagrams illustrating exemplary imaging slab setting performed by the setting function according to the first embodiment;

FIG. 11 is a diagram illustrating exemplary imaging slab setting performed by the setting function according to the first embodiment;

FIG. 12 is a diagram illustrating exemplary imaging slab setting performed by the setting function according to the first embodiment;

FIG. 13 is a diagram illustrating exemplary imaging slab setting performed by the setting function according to the first embodiment;

FIG. 14 is a diagram illustrating exemplary data acquisition performed by an acquisition function according to the first embodiment;

FIG. 15 is a diagram illustrating exemplary data acquisition performed by the acquisition function according to the first embodiment;

FIG. 16 is a diagram illustrating exemplary signal existence determination performed by a determination function according to the first embodiment;

FIG. 17 is a diagram illustrating exemplary LVO existence determination performed by the determination function according to the first embodiment;

FIG. 18 is a diagram illustrating exemplary LVO existence determination performed by the determination function according to a first modification of the first embodiment;

FIG. 19 is a diagram illustrating exemplary LVO existence determination performed by the MRI apparatus according to a second embodiment;

FIGS. 20A and 20B are diagrams illustrating exemplary LVO existence determination performed by the MRI apparatus according to a third embodiment; and

FIG. 21 is a diagram illustrating exemplary LVO existence determination performed by the MRI apparatus according to the third embodiment.

### DETAILED DESCRIPTION

An MRI apparatus according to an embodiment includes a setting unit, an acquisition unit, a generation unit, and a determination unit. The setting unit sets an imaging region and a labeling region based on at least a blood vessel structure, the imaging region being a region of a matrix of a plurality of divided voxels including different blood vessel regions, respectively, the labeling region being a region to which a labeling pulse for labeling blood flowing into the imaging region is applied. The acquisition unit acquires data of the imaging region by applying the labeling pulse to the labeling region by using an arterial spin labeling (ASL) method. The generation unit generates an image based on the data. The determination unit determines anomaly of the blood vessel regions by comparing signal values of the voxels included in the image.

Embodiments of an MRI apparatus according to the present application will be described below in detail with reference to the accompanying drawings.

### First Embodiment

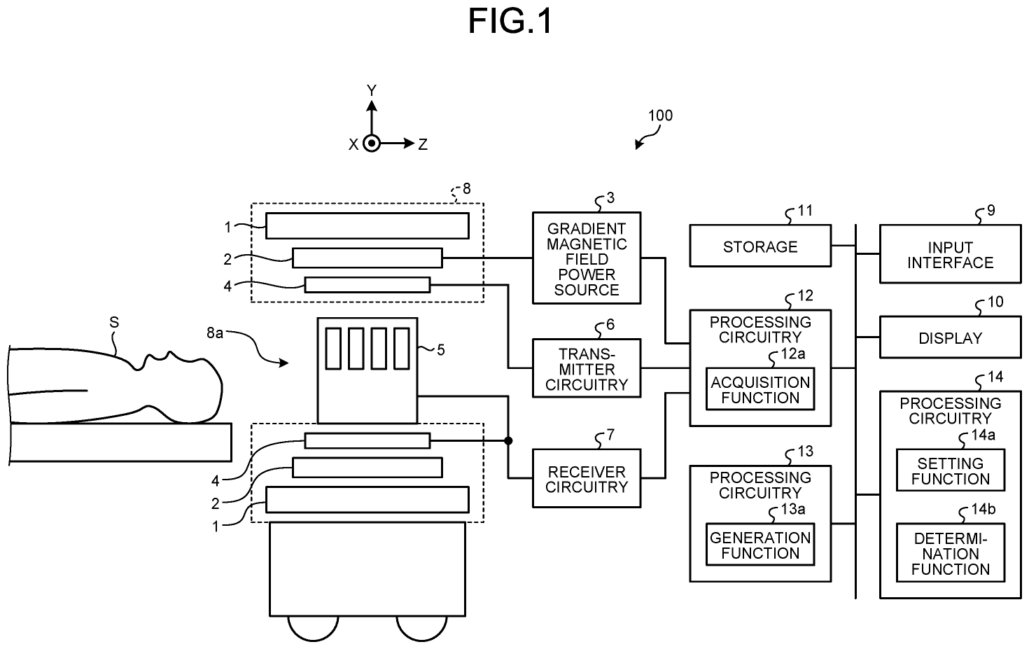

FIG. 1 is a diagram illustrating an exemplary configuration of an MRI apparatus according to a first embodiment.

For example, as illustrated in FIG. 1, an MRI apparatus 100 includes a static magnetic field magnet 1, a gradient coil 2, a gradient magnetic field power source 3, a transmitter-receiver coil 4, a local coil 5, transmitter circuitry 6, receiver circuitry 7, a gantry 8, an input interface 9, a display 10, a storage 11, and processing circuitries 12 to 14.

The static magnetic field magnet 1 generates a static magnetic field in an image capturing space in which a subject S is disposed. For example, the static magnetic field magnet 1 includes a pair of magnets disposed to face each other in the vertical direction with the image capturing space interposed therebetween and generates a static magnetic field in the vertical direction in the image capturing space. Alternatively, for example, the static magnetic field magnet 1 may include a pair of magnets disposed to face each other in the horizontal direction with the image capturing space interposed therebetween and generate a static magnetic field in the horizontal direction in the image capturing space. For example, the static magnetic field magnet 1 is achieved as a permanent magnet.

The gradient coil 2 is disposed inside the static magnetic field magnet 1 and generates a gradient magnetic field in the image capturing space. For example, the gradient coil 2 includes an X coil, a Y coil, and a Z coil corresponding to an X axis, a Y axis, and a Z axis, respectively, orthogonal to one another, and each coil generates a gradient magnetic field having a magnetic field intensity that linearly changes along the corresponding axis. The Y axis is set to match the direction of a static magnetic field generated by the static magnetic field magnet 1, the Z axis is set to match a direction in which the subject S is inserted into the image capturing space, and the X axis is set to match a direction orthogonal to each of the Y axis and the Z axis. Accordingly, the X axis, the Y axis, and the Z axis constitute an apparatus coordinate system unique to the MRI apparatus 100.

The gradient magnetic field power source 3 supplies current to the gradient coil 2 to generate a gradient magnetic field in the image capturing space. Specifically, the gradient magnetic field power source 3 individually supplies current to the X coil, the Y coil, and the Z coil of the gradient coil 2 to generate, in the image capturing space, a gradient magnetic field that linearly changes in each of a readout direction, a phase encode direction, and a slice direction orthogonal to one another. Hereinafter, the gradient magnetic field in the readout direction is referred to as a readout gradient magnetic field, the gradient magnetic field in the phase encode direction is referred to as a phase encode gradient magnetic field, and the gradient magnetic field in the slice direction is referred to as a slice gradient magnetic field.

The readout gradient magnetic field, the phase encode gradient magnetic field, and the slice gradient magnetic field are each superimposed on a static magnetic field generated by the static magnetic field magnet 1 to provide spatial position information to a magnetic resonance signal generated from the subject S. Specifically, the readout gradient magnetic field provides position information in the readout direction to the magnetic resonance signal by changing the frequency of the magnetic resonance signal in accordance with the position in the readout direction. The phase encode gradient magnetic field provides position information in the phase encode direction to the magnetic resonance signal by changing the phase of the magnetic resonance signal in the phase encode direction. The slice gradient magnetic field provides position information in the slice direction to the magnetic resonance signal. For example, when an image capturing region is a slice region (2D image capturing), the slice gradient magnetic field is used to determine the direction and thickness of the slice region and the number of slice regions. When the image capturing region is a volume region (3D image capturing), the slice gradient magnetic field is used to change the phase of the magnetic resonance signal in accordance with the position in the slice direction. Accordingly, an axis in the readout direction, an axis in the phase encode direction, and an axis in the slice direction constitute a logical coordinate system for defining a slice region or volume region as an image capturing target.

The transmitter-receiver coil 4 is disposed inside the gradient coil 2, applies a radio frequency (RF) magnetic field to the subject S disposed in the image capturing space, and receives a magnetic resonance signal generated from the subject S by the RF magnetic field. Specifically, the transmitter-receiver coil 4 applies an RF magnetic field to the subject S disposed in the image capturing space based on an RF pulse supplied from the transmitter circuitry 6. The transmitter-receiver coil 4 receives a magnetic resonance signal generated from the subject S due to influence of the RF magnetic field and outputs the received magnetic resonance signal to the receiver circuitry 7.

The local coil 5 receives a magnetic resonance signal generated from the subject S. Specifically, the local coil 5 is prepared for each site of the subject S and mounted on a target site when image capturing is performed. Then, the local coil 5 receives a magnetic resonance signal generated from the target site due to influence of an RF magnetic field applied by the transmitter-receiver coil 4 and outputs the received magnetic resonance signal to the receiver circuitry 7. The local coil 5 may further have a function to apply an RF magnetic field to the subject S. In this case, the local coil 5 is connected to the transmitter circuitry 6 and applies an RF magnetic field to the subject S based on an RF pulse supplied from the transmitter circuitry 6. For example, the local coil 5 is a surface coil or a phased array coil formed by combining a plurality of surface coils as coil elements.

The transmitter circuitry 6 outputs, to the transmitter-receiver coil 4, an RF pulse corresponding to the Larmor frequency unique to a target nucleus placed in a static magnetic field. Specifically, the transmitter circuitry 6 includes a pulse generator, an RF generator, a modulator, and an amplifier. The pulse generator generates an RF pulse waveform. The RF generator generates an RF signal at a resonance frequency. The modulator generates an RF pulse by modulating the amplitude of the RF signal generated by the RF generator with the waveform generated by the pulse generator. The amplifier amplifies the RF pulse generated by the modulator and outputs the amplified RF pulse to the transmitter-receiver coil 4.

The receiver circuitry 7 generates magnetic resonance data based on a magnetic resonance signal output from the transmitter-receiver coil 4 or the local coil 5 and outputs the generated magnetic resonance data to the processing circuitry 12. For example, the receiver circuitry 7 includes a selector, a prior-stage amplifier, a phase wave detector, and an analog/digital (A/D) converter. The selector selectively inputs a magnetic resonance signal output from the transmitter-receiver coil 4 or the local coil 5. The prior-stage amplifier amplifies the magnetic resonance signal output from the selector. The phase wave detector detects the phase of the magnetic resonance signal output from the prior-stage amplifier. The A/D converter generates magnetic resonance data by converting an analog signal output from the phase wave detector into a digital signal, and outputs the generated magnetic resonance data to the processing circuitry 12. Not every processing performed by the receiver circuitry 7 in the above description does not necessarily need to be performed by the receiver circuitry 7, and part of the processing (for example, the processing by the A/D converter) may be performed by the transmitter-receiver coil 4 or the local coil 5.

The gantry 8 has an opening part 8a forming the image capturing space and supports the static magnetic field magnet 1, the gradient coil 2, and the transmitter-receiver coil 4 around the opening part 8a.

The input interface 9 receives an operation to input various instructions and various kinds of information from an operator. Specifically, the input interface 9 is connected to the processing circuitry 14, converts the input operation received from the operator into an electric signal, and outputs the electric signal to the processing circuitry 14. For example, the input interface 9 is achieved as a device for performing setting of an image capturing condition and a region of interest (ROI) and the like, such as a trackball, a switch button, a mouse, a keyboard, a touchpad to which an input operation is performed through a touch on an operation surface, a touch screen into which a display screen and a touchpad are integrated, a non-contact input circuitry using an optical sensor, or a voice input circuitry. In the present specification, the input interface 9 is not limited to a device including a physical operation member such as a mouse or a keyboard. Examples of the input interface 9 include an electric-signal processing circuitry that receives, from an external input instrument provided separately from the apparatus, an electric signal corresponding to an input operation, and outputs the electric signal to a control circuitry.

The display 10 displays various kinds of information. Specifically, the display 10 is connected to the processing circuitry 14, converts data of various kinds of information transferred from the processing circuitry 14 into a display electric signal, and outputs the display electric signal. For example, the display 10 is achieved as a liquid crystal monitor, a cathode ray tube (CRT) monitor, or a touch panel.

The storage 11 stores various kinds of data. Specifically, the storage 11 is connected to the processing circuitries 12 to 14 and stores various kinds of data input and output by each processing circuitry. For example, the storage 11 is achieved as a semiconductor memory element such as a random access memory (RAM) or a flash memory, a hard disk, or an optical disk.

The processing circuitry 12 has an acquisition function 12a. The acquisition function 12a acquires k-space data by executing various pulse sequences. Specifically, the acquisition function 12a executes various pulse sequences by driving the gradient magnetic field power source 3, the transmitter circuitry 6, and the receiver circuitry 7 in accordance with sequence execution data output from the processing circuitry 14. The sequence execution data is data indicating a pulse sequence and is information defining, for example, a timing at which the gradient magnetic field power source 3 supplies current to the gradient coil 2, the strength of the supplied current, a timing at which the transmitter circuitry 6 supplies an RF pulse to the transmitter-receiver coil 4, the strength of the supplied RF pulse, and a timing at which the receiver circuitry 7 samples a magnetic resonance signal. Then, the acquisition function 12a receives magnetic resonance data output from the receiver circuitry 7 as a result of execution of a pulse sequence and stores the received magnetic resonance data in the storage 11. The magnetic resonance data stored in the storage 11 is provided with position information in the readout direction, the phase encode direction, and the slice direction by the respective gradient magnetic fields described above, and stored as k-space data corresponding to the two-dimensional or three-dimensional k space.

The processing circuitry 13 has a generation function 13a. The generation function 13a generates an image from the k-space data acquired by the processing circuitry 12. Specifically, the generation function 13a reads the k-space data acquired by the processing circuitry 12 from the storage 11 and generates a two-dimensional or three-dimensional image by providing the read k-space data with reconstruction processing such as Fourier transform. Then, the generation function 13a stores the generated image in the storage 11.

The processing circuitry 14 performs entire control of the MRI apparatus 100 by controlling each component included in the MRI apparatus 100. Specifically, the processing circuitry 14 displays, on the display 10, a graphical user interface (GUI) for receiving an operation to input various instructions and various kinds of information from the operator, and controls each component included in the MRI apparatus 100 in accordance with such an input operation received through the input interface 9. For example, the processing circuitry 14 generates sequence execution data based on an image capturing condition input by the operator and outputs the generated sequence execution data to the processing circuitry 12 to acquire k-space data. For example, the processing circuitry 14 controls the processing circuitry 13 to reconstruct an image from the k-space data acquired by the processing circuitry 12. For example, the processing circuitry 14 reads an image from the storage 11 in accordance with a request from the operator, and displays the read image on the display 10.

In the present embodiment, the processing circuitry 14 has a setting function 14a and a determination function 14b. The setting function 14a and the determination function 14b will be described later in detail.

The processing circuitries 12 to 14 described above are achieved as, for example, a processor. In this case, any processing function of each processing circuitry is stored as, for example, a computer-executable program in the storage 11. Each processing circuitry reads a computer program from the storage 11 and executes the computer program to achieve any processing function corresponding to the computer program. In other words, each processing circuitry having read a computer program has each corresponding function indicated in the processing circuitry in FIG. 1.

The following description assumes that the processing circuitries are achieved by one processor, but the embodiment is not limited thereto. The processing circuitries may be configured as a plurality of independent processors in combination, and each processing function may be achieved by the corresponding processor executing a computer program. Each processing function of each processing circuitry may be achieved in a manner distributed or integrated to one or a plurality of processing circuitries as appropriate. In the description of the example illustrated in FIG. 1, one storage 11 stores computer programs corresponding to the processing functions, but a plurality of storages may be disposed in a distributed manner and each processing circuitry may read the corresponding computer program from an individual storage.

The exemplary configuration of the MRI apparatus 100 according to the present embodiment is described above. In the MRI apparatus 100 according to the present embodiment with such a configuration, the static magnetic field magnet 1 generates a static magnetic field having a magnetic field intensity (for example, 0.064 T (Tesla)) lower than the magnetic field intensity (for example, 1.5 T or 3 T) of a typical MRI apparatus that is installed at a hospital or the like. Such an MRI apparatus of a low magnetic field intensity normally can operate with a commercial power source and has a compact apparatus size, and thus is called a portable MRI.

Recently, it has been discussed to mount such a portable MRI on an ambulance and use the portable MRI for solutions in the ambulance. For example, in an examination of cerebral infarction, it is important to specify LVO existence in the anterior circulation system at an early point after the start of symptoms. Thus, it has been discussed to determine LVO existence in an ambulance by using a portable MRI.

However, a portable MRI, which typically has a low magnetic field intensity, has constraints such as a low TOF effect, a long image capturing time, a low resolution, and a low SNR. Thus, with a portable MRI, it is difficult to capture an image of a blood vessel by using a typical TOF method as an MRA method, and accordingly, it is difficult to determine LVO existence.

To solve such a problem, the MRI apparatus 100 according to the present embodiment is configured to be able to determine anomaly of a blood vessel with a low magnetic field intensity by using the ASL method, which can excellently capture an image of a blood vessel with a magnetic field intensity lower than that by the TOF method.

The ASL method generates an image of a blood vessel in which an image of any background tissue is suppressed by performing data acquisition in a tag mode and a control mode and differentiating images generated from the data acquired in the modes. In the tag mode, data of an imaging region set to include a blood vessel as an image capturing target is acquired when a predetermined time has elapsed after a labeling pulse is applied to blood flowing into the imaging region. In the control mode, data of the imaging region is acquired with no labeling pulse application or with a different application position from that in the tag mode.

Specifically, in the present embodiment, the setting function 14a of the processing circuitry 14 sets an imaging region and a labeling region based on at least a blood vessel structure, the imaging region being a region of a matrix of a plurality of divided voxels including different blood vessel regions, respectively, the labeling region being a region to which a labeling pulse for labeling blood flowing into the imaging region is applied. The acquisition function 12a of the processing circuitry 12 acquires data of the imaging region by applying the labeling pulse to the labeling region by using the ASL method. The generation function 13a of the processing circuitry 13 generates an image based on the data acquired by the acquisition function 12a. Then, the determination function 14b of the processing circuitry 14 determines anomaly of the blood vessel regions by comparing signal values of the voxels included in the image generated by the generation function 13a.

The setting function 14a is an example of the setting unit. The acquisition function 12a is an example of the acquisition unit. The generation function 13a is an example of the generation unit. The determination function 14b is an example of the determination unit.

For example, the setting function 14a detects the boundary between the different blood vessel regions based on the blood vessel structure and sets the imaging region and the labeling region based on the detected boundary. For example, the setting function 14a determines a T1 value of the blood in accordance with a magnetic field intensity and sets the imaging region and the labeling region based on the determined T1 value.

The setting function 14a calculates a labeling sustained duration of the blood after the labeling pulse application based on the position of the boundary between the different blood vessel regions and the T1 value of the blood and sets the imaging region and the labeling region based on the calculated labeling sustained duration. The setting function 14a calculates a labeling travel distance based on the labeling sustained duration and the speed of blood current and sets the imaging region and the labeling region based on the calculated labeling travel distance.

The setting function 14a sets the labeling region by determining the position of the labeling region based on the position of the boundary between the different blood vessel regions and the labeling travel distance. The setting function 14a sets the imaging region by determining the position and size of the imaging region based on the position of the boundary between the different blood vessel regions and the labeling travel distance.

The acquisition function 12a acquires data of the imaging region in a plurality of time phases, the generation function 13a generates an image for each time phase, and the determination function 14b determines anomaly of the blood vessel region based on temporal change of signal values of the voxels included in each image. The setting function 14a calculates the position of the boundary between the different blood vessel regions and a time phase and a time interval in which data of the imaging region is acquired, based on a position at which a blood vessel contacts an end face of the labeling region, the thickness of the labeling region, the speed of blood current, and the blood vessel structure, and the acquisition function 12a acquires data of the imaging region based on the position of the boundary, the time phase, and the time interval.

The different blood vessel regions are, for example, a large vessel and a non-large vessel in a brain.

Processing performed by the MRI apparatus 100 according to the present embodiment will be specifically described below with an example in which LVO existence in an artery in the anterior circulation system of a brain is determined. Hereinafter, a large vessel is referred to as an LV, and a non-large vessel is referred to as a non-LV. The following description will be made with an example in which the imaging region and the labeling region are each a slab-shaped region. The slab-shaped imaging region is referred to as an imaging slab, and the slab-shaped labeling region is referred to as a labeling slab.

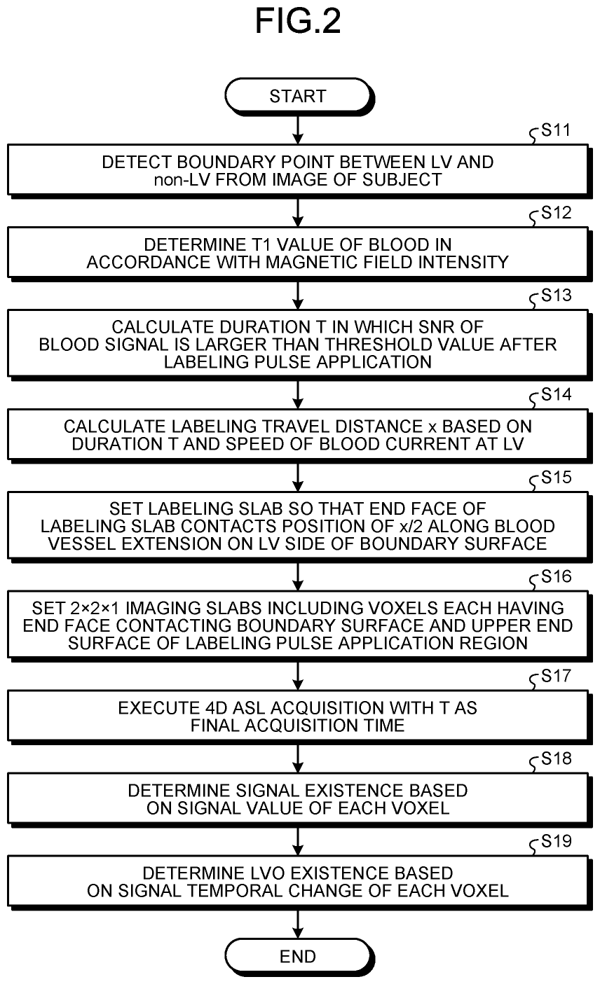

FIG. 2 is a flowchart illustrating the procedure of processing performed by the MRI apparatus 100 according to the first embodiment.

For example, as illustrated in FIG. 2, first, the setting function 14a detects a boundary point between the LV and the non-LV from an image of a subject based on the blood vessel structure (step S11).

Specifically, the setting function 14a detects, for each of right and left arteries of the anterior circulation system, the boundary point between the LV and the non-LV from the image of the subject based on the blood vessel structure.

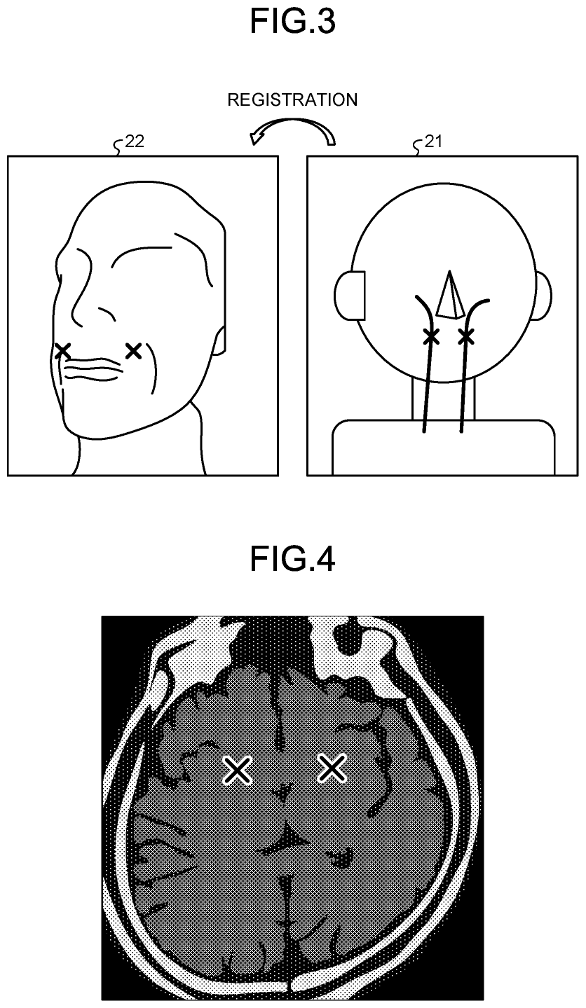

FIGS. 3 to 6 are diagrams illustrating exemplary detection of the boundary between the LV and the non-LV, which is performed by the setting function 14a according to the first embodiment.

For example, as illustrated in FIG. 3, the setting function 14a specifies the positions of the LV and the non-LV by registering a stereoscopic model 21 of a head including blood vessels and an image 22 of the subject, which is captured by a camera or the like. In this case, for example, the setting function 14a registers the surface shape of the head in the stereoscopic model 21 and the surface shape of the head in the image 22 of the subject. Then, the setting function 14a detects each boundary point (point illustrated with a cross in FIG. 3) based on the specified positions of the LV and the non-LV.

Alternatively, for example, as illustrated in FIG. 4, the setting function 14a may specify the positions of the LV and the non-LV by registering an image captured by the MRI apparatus 100 in advance and a stereoscopic model of a head including blood vessels and may detect each boundary point (point illustrated with a cross in FIG. 4) based on the specified positions of the LV and the non-LV.

Alternatively, for example, the setting function 14a, may detect each boundary point by specifying the positions of the LV and the non-LV from an image of the subject, which is captured by a camera or the MRI apparatus 100, by using a machine learning model that detects a brain blood vessel.

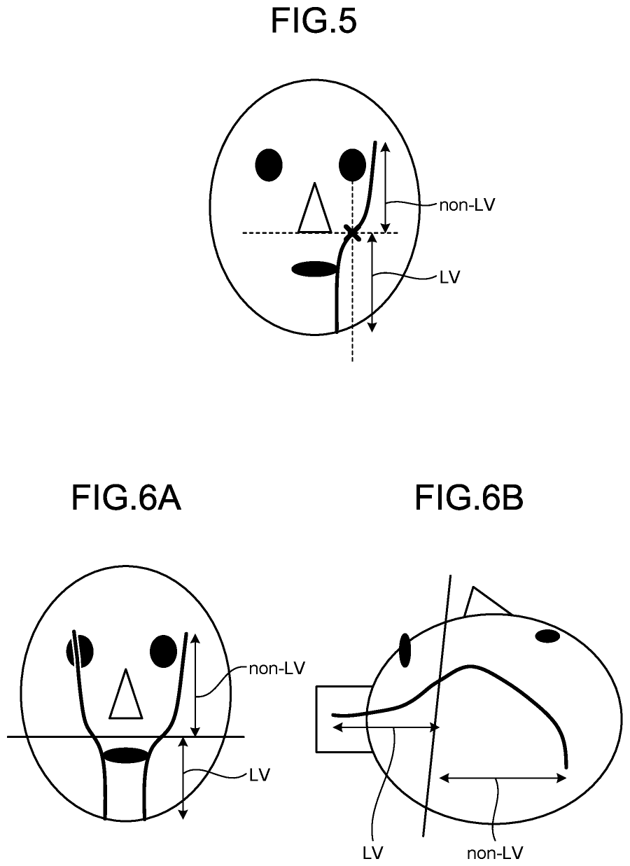

Alternatively, for example, as illustrated in FIG. 5, the setting function 14a may detect the boundary point between the LV and the non-LV by detecting a feature point corresponding to the blood vessel structure from an image of the subject, which is captured by a camera or the MRI apparatus 100. For example, the setting function 14a detects, from the image of the subject, an axial plane passing below the nose and a sagittal plane passing through the center of an eye. The setting function 14a detects, on a line on which the detected axial plane and sagittal plane intersect each other, a point inside the head by a predetermined length (for example, 3 cm) from the surface of the head, and determines the detected point as the boundary point between the LV and the non-LV.

Alternatively, for example, as illustrated in FIGS. 6A and 6B, the setting function 14a may detect a section passing through each boundary point between the LV and the non-LV. Alternatively, for example, the setting function 14a may detect a volume region passing through each boundary point between the LV and the non-LV.

Returning to FIG. 2, the setting function 14a subsequently determines the T1 value of blood in accordance with a magnetic field intensity (step S12).

For example, when a fixed magnetic field intensity is used in the MRI apparatus 100, the setting function 14a determines the T1 value of blood by referring to information of the T1 value stored in the storage 11 in advance.

Alternatively, for example, when a changeable magnetic field intensity is used in the MRI apparatus 100, the setting function 14a may determine the T1 value of blood based on a magnetic field intensity by referring to data representing the relation between the magnetic field intensity and the T1 value, which is stored in the storage 11 in advance. Alternatively, the setting function 14a may determine the T1 value of blood based on a magnetic field intensity by calculating the T1 value in accordance with an expression representing the relation between the magnetic field intensity and the T1 value, which is defined in advance.

Subsequently, the setting function 14a calculates a labeling sustained duration T of blood by calculating, based on the position of the boundary between the LV and the non-LV and the T1 value of blood, a duration in which the SNR of a blood signal is larger than a threshold value after the labeling pulse application (step S13).

For example, a signal intensity Mz(t) when time t has elapsed after the labeling pulse application is expressed by Expression (1) below.

Mz(t)=M0{1−2exp(−t/T1)} (1)

Thus, for example, the setting function 14a calculates the labeling sustained duration T based on Expression (1) above. In this case, the setting function 14a uses an SNR threshold value to estimate the labeling sustained duration T at an iso-chromat level. For example, when an SNR (Mz/noise level) to be assured is 1.1, the setting function 14a calculates time t at which SNR>1.1 holds, and determines the calculated time t as the labeling sustained duration T. In this case, the noise level may be input by the operator, may be stored in the storage 11 in advance, or may be estimated by performing scanning in advance. In a case in which the noise level is estimated by scanning, a pulse sequence same as that in the actual image capturing may be executed twice without labeling and the noise level may be calculated by differentiating acquired data.

The above-described SNR calculation is performed at the iso-chromat level and does not use the actual SNR. Thus, for example, the setting function 14a may calculate the labeling sustained duration T by calculating the SNR based on signal values when it is assumed that a matrix of voxels as imaging slabs is 2×2×1 and one voxel is filled with the non-LV. For example, in Expression (1) above, M0 is defined based on the density of a blood vessel included in the voxel. The value M0 may be determined based on an empirical value or may be estimated by performing scanning under the same condition in advance.

Subsequently, the setting function 14a calculates a labeling travel distance x based on the labeling sustained duration T and the speed of blood current at an LV (step S14).

For example, the setting function 14a calculates the labeling travel distance x by x=v×T, where v represents the speed of blood current of blood flowing through the LV. The labeling travel distance x is a maximum distance by which the blood signal remains. In this case, the speed of blood current may be determined and stored in the storage 11 in advance, may be measured by an ultrasonic wave diagnostic apparatus, or may be measured by executing another pulse sequence (for example, a pulse sequence by a phase contrast method) at the MRI apparatus 100. For example, when determined in advance, the speed of blood current is set to be 200 cm/s approximately, which is a typical speed at an internal carotid artery.

Alternatively, for example, the setting function 14a may calculate the labeling travel distance x by x=v(r)×dT, where r represents a position in a blood vessel and v(r) represents the speed of blood current at the position r.

Subsequently, the setting function 14a sets the labeling slab based on the labeling travel distance x so that an end face of the labeling slab contacts the position of x/2 along blood vessel extension on the LV side of a boundary surface (step S15).

Specifically, the setting function 14a sets the labeling slab by determining the position of the labeling slab based on the position of the boundary between the LV and the non-LV and the labeling travel distance x for each of the right and left arteries of the anterior circulation system.

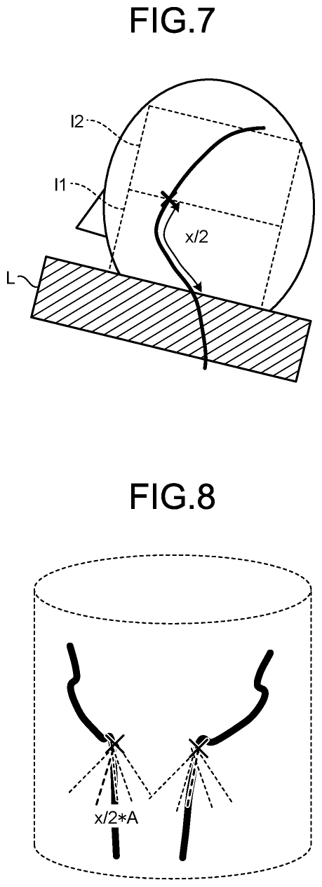

FIGS. 7 to 9 are diagrams illustrating exemplary labeling slab setting performed by the setting function 14a according to the first embodiment.

For example, as illustrated in FIG. 7, the setting function 14a sets the position of a labeling slab L so that the upper end surface thereof is located at a position separated by x/2 along blood vessel extension on a proximal side of a boundary surface passing through the boundary points between the LV and the non-LV in the right and left arteries of the anterior circulation system.

Accordingly, when imaging slabs I1 and I2 are set downstream of the labeling slab L to include the LV and the non-LV by x/2, respectively, the blood signals of the LV and the non-LV can be sustained in the imaging slabs I1 and I2.

In this case, for example, the setting function 14a specifies blood vessel extension based on a stereoscopic model used to detect each boundary point between the LV and the non-LV or an image captured by the MRI apparatus 100 in advance. Alternatively, for example, the setting function 14a may specify blood vessel extension by using an algorithm defined to automatically specify blood vessel extension based on the shape of a head.

Alternatively, for example, as illustrated in FIG. 8, the setting function 14a may calculate a linear distance by x/2×A by using an average distortion ratio A of the LV without using the blood vessel structure and may set the labeling slab so that the upper end surface of the labeling slab is located at a position separated by the linear distance on the proximal side of the boundary point between the LV and the non-LV. The distortion ratio A is the ratio of the linear distance relative to a curved distance between points.

The ASL method includes a signal targeting with alternating radiofrequency (STAR) system and a flow-sensitive alternating inversion recovery (FAIR) system. The STAR system applies a labeling pulse to an upstream part of blood flowing into each imaging slab in the tag mode. The FAIR system applies a labeling pulse to an upstream part of blood flowing into each imaging slab and a range including the imaging slab in the tag mode, and applies a control pulse similar to the labeling pulse to the range including the imaging slab in the control mode.

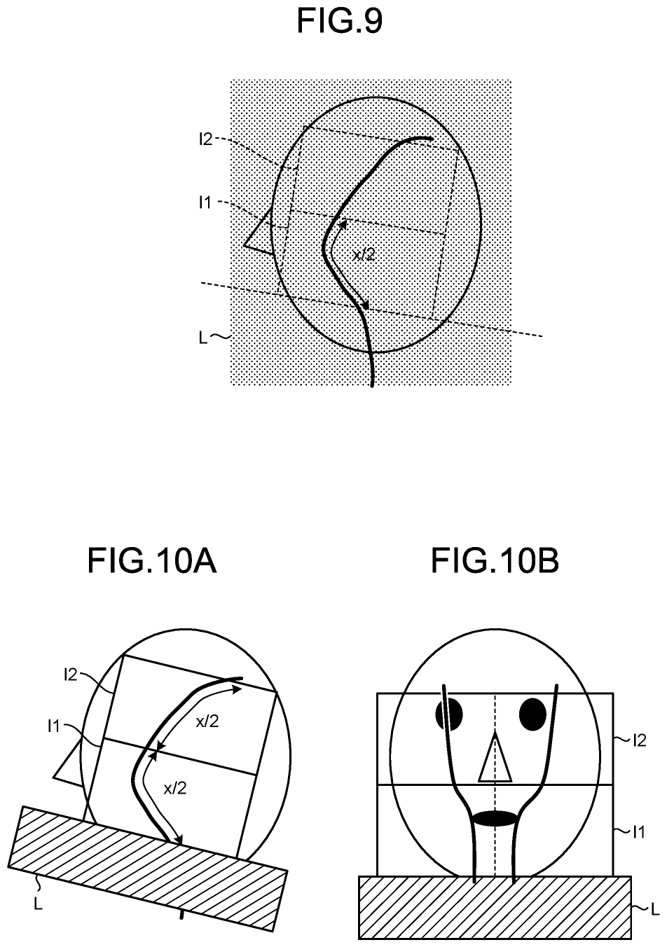

For example, when the STAR system is used, the setting function 14a sets the labeling slab by the above-described method. For example, when the FAIR system is used, as illustrated in FIG. 9, the setting function 14a sets the imaging slabs I1 and I2 and the labeling slab L so that the lower end surface of the imaging slabs is located at a position separated by x/2 along blood vessel extension on the proximal side of the boundary point between the LV and the non-LV. In this case, the position at which the lower end surface of the imaging slabs is located does not necessarily need to be a position separated by x/2 along blood vessel extension from the boundary point, but may be a position separated by a distance shorter than x/2 along blood vessel extension from the boundary point.

Returning to FIG. 2, subsequently, the setting function 14a sets 2×2×1 imaging slabs including voxels each having an end face contacting the boundary surface and the upper end surface of the labeling pulse application region (step S16).

Specifically, with respect to the right and left arteries of the anterior circulation system, the setting function 14a sets imaging slabs in a matrix of four divided voxels, namely, a voxel including the right LV, a voxel including the right non-LV, a voxel including the left LV, and a voxel including the left non-LV.

In this case, the setting function 14a sets each imaging slab by determining the position and size of the imaging slab based on the position of the boundary between the LV and the non-LV and the labeling travel distance x in each of the right and left arteries of the anterior circulation system.

FIGS. 10A to 13 are diagrams illustrating exemplary imaging slab setting performed by the setting function 14a according to the first embodiment.

For example, as illustrated in FIGS. 10A and 10B, the setting function 14a sets an imaging slab I1 including an LV by determining the position and size of the imaging slab so that the lower end surface thereof is located at a position separated by x/2 in a blood vessel direction on the proximal side of the boundary surface passing through the boundary point between the LV and the non-LV in each of the right and left arteries of the anterior circulation system. In addition, the setting function 14a sets an imaging slab I2 including a non-LV by determining the position and size of the imaging slab so that the upper end surface thereof is located at a position separated by x/2 in the blood vessel direction on the distal side of the boundary surface passing through the boundary point between the LV and the non-LV in each of the right and left arteries of the anterior circulation system. In this case, the positions at which the lower end surface of the imaging slab I1 and the upper end surface of the imaging slab I2 are located do not necessarily need to be positions separated by x/2 along blood vessel extension from the boundary points but may be positions separated by distances shorter than x/2 along blood vessel extension from the boundary points.

The setting function 14a determines the position and size of each imaging slab so that the upper end surface of the imaging slab I1 contacts the lower end surface of the imaging slab I2 and the lower end surface of the imaging slab I1 is positioned closest to the upper end surface of the labeling slab L.

Accordingly, the two imaging slabs I1 and I2 are set downstream of the labeling slab L to include, by x/2, the LV and the non-LV, respectively, in each of the right and left arteries of the anterior circulation system.

Then, the setting function 14a sets the imaging slabs so that the imaging slabs I1 and I2 each include two voxels divided in the right-left direction at the median plane of the subject. Accordingly, the setting function 14a sets the imaging slabs I1 and I2 as imaging slabs in a 2×2×1 matrix of four divided voxels, namely, the voxel including the right LV, the voxel including the right non-LV, the voxel including the left LV, and the voxel including the left non-LV. The expression “2×2×1” means that rectangular parallelepiped voxels are arrayed on two rows and two columns in a coronal plane and arrayed on two rows and one column in a sagittal plane.

The above description is made with an example in which the position and size of each imaging slab are set so that the lower end surface of the imaging slab I1 is positioned closest to the upper end surface of the labeling slab L, but the method of setting imaging slabs is not limited to the example.

For example, as illustrated in FIG. 11, the setting function 14a may calculate a regression line for a curved line structure obtained by the core line of a blood vessel, and may set each imaging slab to be orthogonal to the regression line. In this case, for example, the setting function 14a may calculate a regression line for each of the LV and the non-LV and set each imaging slab, or may calculate a regression line for one of the LV and the non-LV and set each imaging slab.

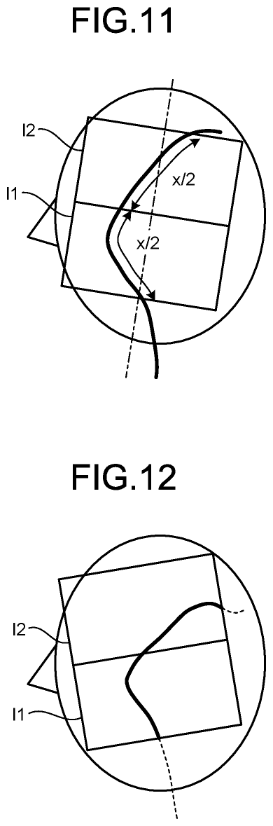

Alternatively, for example, as illustrated in FIG. 12, the setting function 14a may set imaging slabs by setting slabs (obliques) tilted to include the LV and the non-LV, respectively, by the same length from a boundary surface passing through the boundary point between the LV and the non-LV.

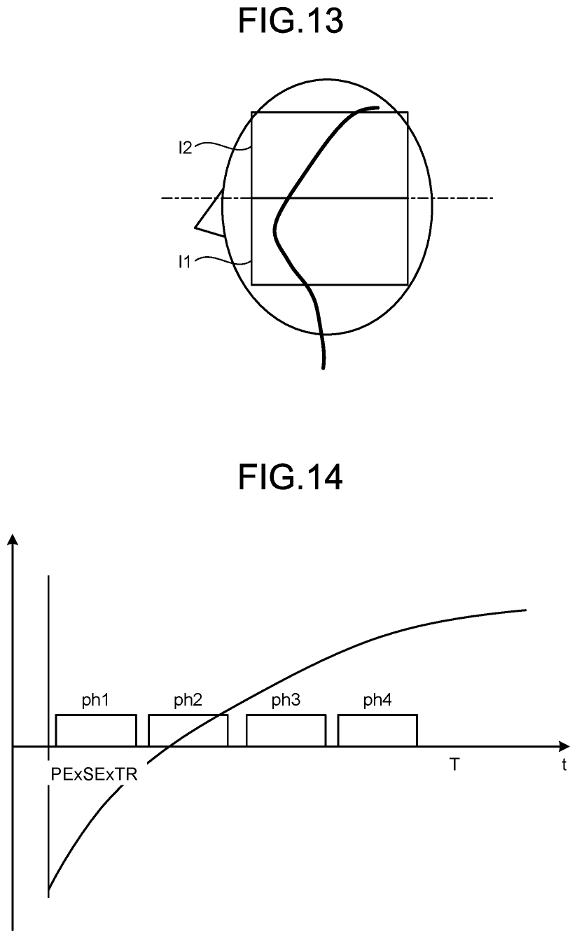

Alternatively, for example, as illustrated in FIG. 13, the setting function 14a may assume that a blood vessel extends in a relative positional relation with a straight line, and may set each imaging slab based on an orbitomeatal (OM) line (line illustrated with a dashed and single-dotted line in FIG. 13) or an anterior commissure (AC)-posterior commissure (PC) line. The OM line is a line connecting the orbital center and the external auditory foramen center. The AC-PC line is a line connecting the anterior commissure and the posterior commissure. Alternatively, for example, the setting function 14a may set each imaging slab by receiving the position and size of the slab from the operator.

In any of the examples, a voxel included in each imaging slab is desirably set to avoid duplication with the labeling slab. Moreover, the imaging slab and the labeling slab do not necessarily need to contact each other, and the size of a voxel included in the imaging slab may be smaller than the size of a voxel included in the labeling slab.

Returning to FIG. 2, subsequently, the acquisition function 12a executes 4D ASL acquisition with T as a final acquisition time, and the generation function 13a generates an image based on acquired data (step S17).

Specifically, the acquisition function 12a acquires data of each imaging slab by applying a labeling pulse to the labeling slab set by the setting function 14a by using the ASL method.

The acquisition function 12a acquires data of each imaging slab in a plurality of time phases. Then, the generation function 13a generates an image for each time phase.

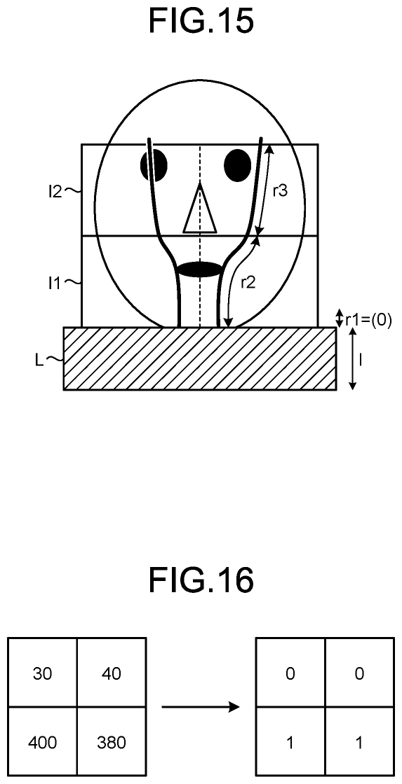

FIGS. 14 and 15 are diagrams illustrating exemplary data acquisition performed by the acquisition function 12a according to the first embodiment.

For example, as illustrated in FIG. 14, after having applied a labeling pulse to the labeling slab, the acquisition function 12a continuously acquires data in a plurality of time phases (ph1, ph2, ph3, and ph4 illustrated in FIG. 14) during the labeling sustained duration T.

As a specific example, in a case of T=500 ms, the acquisition function 12a acquires data of 500 ms/(2×1×3)=83 time phases by using a gradient echo (GRE) method with a phase encode number (PE)=2, a slice encode number (SE)=1, and a repetition time (TR)=3 ms. The number of time phases in which the acquisition function 12a acquires data is not limited thereto but may be less than 83 time phases. The following description will be made with an example in which the acquisition function 12a acquires data of four time phases.

For example, as illustrated in FIG. 15, blood flows into the lower end of the voxel of the imaging slab I1 at t0=r1/v and into the lower end of the voxel of the imaging slab I2 at t1=(r1+r2)/v, where r1 represents the length of the LV along blood vessel extension between the upper end surface of the labeling slab L and the lower end surface of the imaging slab I1 (r1=0 when the lower end surface of the imaging slab I1 contacts the upper end surface of the labeling slab L), and r2 represents the length of the LV along blood vessel extension in the imaging slab I1. Then, the lower end of labeled blood passes through the upper end surface of the voxel of the imaging slab I2 at t3=(r1+r2+r3+l)/v, where l represents the length of the labeling slab L in the up-down direction and r3 represents the length of the non-LV along blood vessel extension in the imaging slab I2.

Specifically, in the present example, the blood signal is lost in the labeling sustained duration T when the lower end of labeled blood reaches the upper end surface of the voxel of the imaging slab I2, but the blood signal is lost at t3 when the size of the imaging slab is small. In this case, no blood is visualized irrespective of data acquisition at t3 or later, and thus data acquisition is unnecessary.

Returning to FIG. 2, subsequently, the determination function 14b determines signal existence based on the signal value of each voxel included in the image generated by the generation function 13a (step S18).

Specifically, the determination function 14b determines signal existence based on the signal values of four voxels included in the image generated by the generation function 13a, namely, the voxel including the right LV, the voxel including the right non-LV, the voxel including the left LV, and the voxel including the left non-LV.

FIG. 16 is a diagram illustrating exemplary signal existence determination performed by the determination function 14b according to the first embodiment.

For example, as illustrated in FIG. 16, by using a predetermined threshold value, for each of the four voxels, the determination function 14b determines that a signal exists (1) when the signal value thereof is equal to or larger than the threshold value, or determines that no signal exists (0) when the signal value is smaller than the threshold value.

In this case, for example, to distinguish variation of the blood signal from variation of a background signal, the determination function 14b may compare the signal values of a voxel among images generated for the respective time phases by the generation function 13a, thereby determining signal existence based on temporal change of the signal value. For example, when change of the signal value of a voxel between the previous and next time phases is larger than a predetermined threshold value, the determination function 14b determines: that no signal exists for the voxel in the previous time phase and a signal exists for the voxel in the next time phase in a case in which the signal value increases; or that a signal exists for the voxel in the previous time phase and no signal exists for the voxel in the next time phase in a case in which the signal value decreases.

Subsequently, the determination function 14b determines LVO existence based on the signal temporal change of each voxel (step S19).

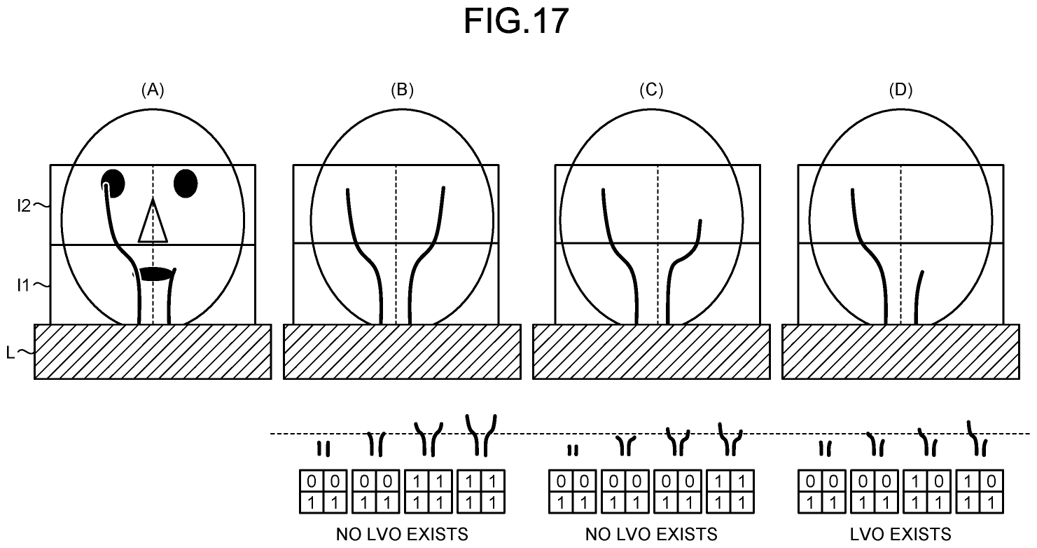

FIG. 17 is a diagram illustrating exemplary LVO existence determination performed by the determination function 14b according to the first embodiment.

For example, as illustrated in portions (A) to (D) of FIG. 17, there is data of four voxels on two rows and two columns in a coronal plane for four time phases. In this case, for example, the determination function 14b determines that an LVO exists when the signal existence is same between the right and left voxels on the LV side (the lower row in FIG. 17) for all time phases and the signal existence is different between the right and left voxels on the non-LV side (the upper row in FIG. 17) at or after a middle time phase (the third time phase in the example of FIG. 17) as illustrated in portion (D) of FIG. 17.

For example, the determination function 14b determines that no LVO exists when the signal existence is same between the right and left voxels on the LV side for all time phases and the signal existence is same between the right and left voxels on the non-LV side for all time phases as illustrated in portions (B) and (C) of FIG. 17.

The procedure of processing performed by the MRI apparatus 100 according to the present embodiment is described above. When the processing circuitries 12 to 14 are achieved by a processor, the processing at steps S11 to S16 is achieved, for example, as the processing circuitry 14 reads a predetermined computer program corresponding to the setting function 14a from the storage 11 and executes the read computer program. The processing at step S17 is achieved, for example, as the processing circuitry 12 and the processing circuitry 13 read predetermined computer programs corresponding to the acquisition function 12a and the generation function 13a, respectively, from the storage 11 and execute the read computer programs. The processing at steps S18 and S19 is achieved, for example, as the processing circuitry 14 reads a predetermined computer program corresponding to the determination function 14b from the storage 11 and executes the read computer program.

As described above, according to the first embodiment, the setting function 14a sets an imaging slab and a labeling slab based on at least the blood vessel structure, the imaging slab being a matrix of a plurality of divided voxels including different blood vessel regions, respectively, the labeling slab being a slab to which a labeling pulse for labeling blood flowing into the imaging slab is applied. The acquisition function 12a acquires data of the imaging slab by applying the labeling pulse to the labeling slab by using the ASL method. The generation function 13a generates an image based on the data acquired by the acquisition function 12a. Then, the determination function 14b determines anomaly of the blood vessel region by comparing the signal values of the voxels included in the image generated by the generation function 13a.

In this manner, according to the first embodiment, it is possible to determine anomaly of a blood vessel with a low magnetic field intensity by using the ASL method, which can excellently capture an image of a blood vessel with a magnetic field intensity lower than that by the TOF method.

Accordingly, for example, it is possible to determine LVO existence in an ambulance by using a portable MRI. Moreover, it is possible to completely automatically determine LVO existence as a solution in an ambulance and determine LVO existence irrespective of experience of an operator and the like.

The embodiment is described above with an example in which LVO existence is determined by detecting the boundary between the LV and the non-LV for an artery in the anterior circulation system of a brain, but an example of anomaly determination for a blood vessel region is not limited thereto.

For example, anomaly of a blood vessel region may be determined for a middle cerebral artery (MCA) by detecting the boundary between an M1 zone and an M2 zone.

Alternatively, for example, anomaly of a blood vessel region may be determined by detecting the boundary between a basilar artery (BA) and a posterior cerebral artery (PCA).

The boundary between blood vessel regions does not necessarily need to be continuous. For example, the M1 zone and an M3 zone in the MCA are such blood vessel regions.

Although the first embodiment is described above, the present embodiment may be achieved with appropriate modifications of part of the above-described configuration.

### First Modification of First Embodiment

For example, the first embodiment is described with an example in which the determination function 14b determines LVO existence after determining signal existence (0 or 1) based on the signal value of each voxel, but the method of determining LVO existence is not limited thereto. For example, the determination function 14b may determine LVO existence directly based on the signal value of each voxel.

FIG. 18 is a diagram illustrating exemplary LVO existence determination performed by the determination function 14b according to a first modification of the first embodiment.

For example, as illustrated in FIG. 18, the determination function 14b determines that an LVO exists when the signal values of the right and left voxels on the LV side (lower side in FIG. 18) are larger than a threshold value as a high value for all time phases and the signal values of the right and left voxels are within a range in which the signal values can be regarded as substantially constant, and in addition, when the signal value of one of the right and left voxels on the non-LV side (upper side in FIG. 18) is smaller than a threshold value as a low value at or after a middle time phase (the third time phase in the example of FIG. 18).

### Second Modification of First Embodiment

For example, the pieces of processing performed by the MRI apparatus 100 described in the first embodiment do not necessarily need to be executed in the order illustrated in FIG. 2. For example, the processing at step S11 and the processing at step S12 may be executed in the reversed order.

Although the first embodiment is described above, embodiments disclosed in the present application are not limited thereto. Other embodiments of the MRI apparatus according to the present application will be described below. The following description of the embodiments will be mainly made on any difference from the first embodiment, and a description of any content common to that of the first embodiment is omitted.

### Second Embodiment

The first embodiment is described with an example in which LVO existence is determined at an artery in the anterior circulation system of a brain, but is not limited thereto. For example, the same embodiment is also applicable to a case in which LVO existence is determined at an artery in the posterior circulation system of a brain. An example of such a case will be described below as a second embodiment.

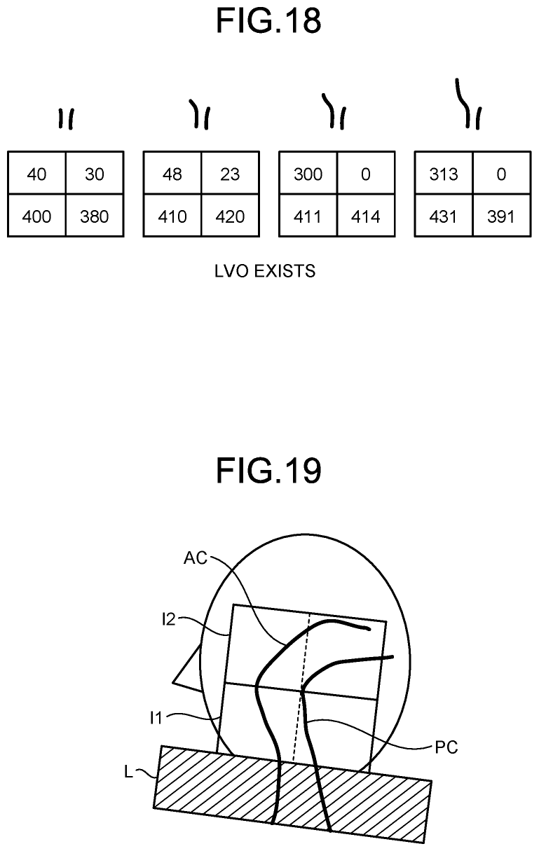

FIG. 19 is a diagram illustrating exemplary LVO existence determination performed by the MRI apparatus 100 according to the second embodiment.

For example, as illustrated in FIG. 19, in the present embodiment, the setting function 14a detects, based on the blood vessel structure, the boundary point between the LV and the non-LV from an image of a subject for each of an artery AC in the anterior circulation system and an artery PC in the posterior circulation system.

The setting function 14a sets imaging slabs as a 2×2×1 matrix of four divided voxels, namely, a voxel including the LV of the artery AC in the anterior circulation system, a voxel including the non-LV of the artery AC in the anterior circulation system, a voxel including the LV of the artery PC in the posterior circulation system, and a voxel including the non-LV of the artery PC in the posterior circulation system. The expression “2×2×1” means that rectangular parallelepiped voxels are arrayed on two rows and two columns in a sagittal plane and arrayed on two rows and one column in a coronal plane.

Then, the determination function 14b determines LVO existence based on the signal values of four voxels included in an image generated by the generation function 13a, namely, the voxel including the LV of the artery AC in the anterior circulation system, the voxel including the non-LV of the artery AC in the anterior circulation system, the voxel including the LV of the artery PC in the posterior circulation system, the voxel including the non-LV of the artery PC in the posterior circulation system.

When an artery in the posterior circulation system is targeted as in the present embodiment, the non-LV of the artery AC in the anterior circulation system exists in the voxel including the non-LV of the artery PC in the posterior circulation system, such as the upper-right voxel in FIG. 19, in some cases because of the blood vessel structure in the brain.

Thus, for example, the determination function 14b compensates a signal value for a voxel in which the non-LV of the artery PC in the posterior circulation system and the non-LV of the artery AC in the anterior circulation system exist in mixture, by estimating and removing a signal value from the artery AC in the anterior circulation system or estimating only a signal value from the artery PC in the posterior circulation system.

For example, the determination function 14b estimates a signal value from the artery PC in the posterior circulation system based on the ratio of blood vessel densities in the voxel. Alternatively, for example, the determination function 14b may estimate, for each time phase, a signal value on the non-LV side from a signal value on the LV side based on the ratio of blood current amounts of the LV and the non-LV. In this case, for example, the determination function 14b acquires the ratio of blood vessel densities or blood current amounts from brain-related anatomical information stored in the storage 11 in advance.

### Third Embodiment

The first embodiment is described with an example in which the setting function 14a sets imaging slabs as a 2×2×1 matrix of four divided voxels, but is not limited thereto. For example, the same embodiment is also applicable to a case in which the number of voxels included in the matrix of imaging slabs is larger than four. An example of such a case will be described below as a third embodiment.

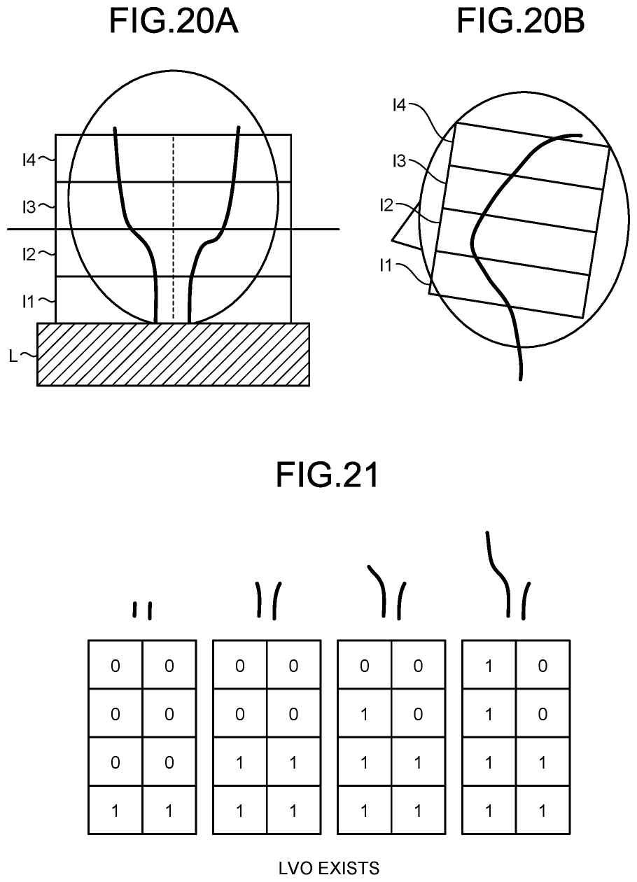

FIGS. 20A to 21 are diagrams illustrating exemplary LVO existence determination performed by the MRI apparatus 100 according to the third embodiment.

For example, as illustrated in FIGS. 20A and 20B, in the present embodiment, the setting function 14a sets four imaging slabs I1 to I4 along blood vessel extension by dividing each of the imaging slabs I1 and I2 in the first embodiment into two.

Then, the setting function 14a sets the imaging slabs so that the imaging slabs I1 to I4 each include two voxels divided in the right-left direction at the median plane of the subject. Accordingly, the setting function 14a sets the imaging slabs I1 to I4 as imaging slabs in a 4×2×1 matrix of eight divided voxels, namely, a voxel including a proximal side part of the right LV, a voxel including a distal side part of the right LV, a voxel including a proximal side part of the right non-LV, a voxel including a distal side part of the right non-LV, a voxel including a proximal side part of the left LV, a voxel including a distal side part of the left LV, a voxel including a proximal side part of the left non-LV, and a voxel including a distal side part of the left non-LV. The expression “4×2×1” means that rectangular parallelepiped voxels are arrayed on four rows and two columns in a coronal plane and arrayed on four rows and one column in a sagittal plane.

For example, consider a case in which the matrix of imaging slabs includes two or more voxels in an HF direction (craniocaudal direction) and the two or more voxels correspond to a phase encode step. In this case, for example, the setting function 14a sets TR so that Expression (2) below is satisfied, where n represents maximum matrix number at which image capturing is possible with the matrix of imaging slabs.

n×1×TR<t3 (2)

In Expression (2), as described above, t3 represents a time when the lower end of labeled blood passes through the upper end surface of the voxel of the imaging slab I2.

For example, when S represents the signal value of the voxel closest to the distal side in the second time phase, the signal value S decreases in accordance with the size of the voxel. Thus, for example, the setting function 14a sets the size of the voxel so that the signal value S satisfies Expression (3) below.

S>threshold value (3)

Then, the determination function 14b determines LVO existence based on the signal values of the eight voxels set by the setting function 14a.

For example, as illustrated in FIG. 21, there is data of the eight voxels on four rows and two columns in a coronal plane for four time phases. In this case, for example, the determination function 14b determines that an LVO exists when the signal existence is same between the right and left voxels on the LV side (two rows on the lower side in FIG. 21) for all time phases and the signal existence is different between the right and left voxels on the non-LV side (two rows on the upper side in FIG. 21) at or after a middle time phase (the third time phase in the example of FIG. 21).

The first to third embodiments of the MRI apparatus 100 according to the present application are described above.

Each embodiment is described above with an example in which the acquisition unit in the present specification is achieved by the acquisition function 12a of the processing circuitry 12, the generation unit in the present specification is achieved by the generation function 13a of the processing circuitry 13, and the setting unit and the determination unit in the present specification are achieved by the setting function 14a and the determination function 14b of the processing circuitry 14, but the embodiment is not limited thereto. For example, the acquisition unit, the generation unit, the setting unit, and the determination unit in the present specification do not necessarily need to be achieved by the acquisition function 12a, the generation function 13a, the setting function 14a, and the determination function 14b described in the embodiment, but may be achieved by hardware only, by software only, or by mixture of hardware and software.

The above description is made with an example in which a “processor” reads a computer program corresponding to each processing function from a storage and executes the read computer program, but the embodiments are not limited thereto. The term “processor” means circuitry such as a central processing unit (CPU), a graphics processing unit (GPU), an application specific integrated circuit (ASIC), or a programmable logic device (for example, a simple programmable logic device (SPLD), a complex programmable logic device (CPLD), or a field programmable gate array (FPGA)). When the processor is, for example, a CPU, the processor achieves each processing function by reading and executing a computer program stored in a storage. When the processor is an ASIC, the computer program is not stored in a storage, but the processing function is directly incorporated as a logic circuitry in circuitry of the processor. Each processor of the present embodiment does not necessarily need to be configured as one circuitry, but may be configured as one processor of a plurality of independent circuitries in combination to achieve the processing functions. Moreover, a plurality of components in FIG. 1 may be integrated into one processor to achieve the processing functions.

Each computer program executed by a processor is incorporated in a read only memory (ROM), a storage, or the like in advance and provided. The computer program may be recorded and provided in a computer-readable storage medium such as a compact disc (CD)-ROM, a flexible disk (FD), CD-recordable (R), or a digital versatile disc (DVD) as a file in a format that is installable or executable on these devices. The computer program may be stored in a computer connected with a network such as the Internet and provided or distributed by downloading through the network. For example, the computer program is configured as a module including each above-described functional component. Each module is loaded onto a main storage device and generated on the main storage device when a CPU reads the computer program from a storage medium such as a ROM and executes the computer program in an actual hardware configuration.

According to at least one embodiment described above, it is possible to determine anomaly of a blood vessel with a low magnetic field intensity.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A magnetic resonance imaging apparatus, comprising:
processing circuitry configured to
set an imaging region and a labeling region based on at least a blood vessel structure, the imaging region being a region of a matrix of a plurality of divided voxels respectively including different blood vessel regions, the labeling region being a region to which a labeling pulse for labeling blood flowing into the imaging region is applied,
acquire data of the imaging region by applying the labeling pulse to the labeling region by using an arterial spin labeling (ASL) method,
generate an image based on the data, and
determine anomaly of the blood vessel regions by comparing signal values of the voxels included in the image.

2. The magnetic resonance imaging apparatus according to claim 1, wherein the processing circuitry determines a T1 value of the blood in accordance with a magnetic field intensity and sets the imaging region and the labeling region based on the determined T1 value.

3. The magnetic resonance imaging apparatus according to claim 1, wherein the processing circuitry detects a boundary between the different blood vessel regions based on the blood vessel structure and sets the imaging region and the labeling region based on the detected boundary.

4. The magnetic resonance imaging apparatus according to claim 3, wherein the processing circuitry calculates a labeling sustained duration of the blood after the labeling pulse application based on a position of the boundary and a T1 value of the blood and sets the imaging region and the labeling region based on the calculated labeling sustained duration.

5. The magnetic resonance imaging apparatus according to claim 4, wherein the processing circuitry calculates a labeling travel distance based on the labeling sustained duration and a speed of blood current and sets the imaging region and the labeling region based on the calculated labeling travel distance.

6. The magnetic resonance imaging apparatus according to claim 5, wherein the processing circuitry sets the labeling region by determining a position of the labeling region based on the position of the boundary and the labeling travel distance.

7. The magnetic resonance imaging apparatus according to claim 5, wherein the processing circuitry sets the imaging region by determining a position and size of the imaging region based on the position of the boundary and the labeling travel distance.

8. The magnetic resonance imaging apparatus according to claim 1, wherein the processing circuitry
acquires data of the imaging region in a plurality of time phases,
generates an image for each of the time phases, and
determines anomaly of the blood vessel region based on temporal change of signal values of the voxels included in each image.

9. The magnetic resonance imaging apparatus according to claim 8, wherein the processing circuitry
calculates a position of a boundary between the different blood vessel regions and a time phase and a time interval in which data of the imaging region is acquired, based on a position at which a blood vessel contacts an end face of the labeling region, a thickness of the labeling region, a speed of blood current, and the blood vessel structure, and
acquires data of the imaging region based on the position of the boundary, the time phase, and the time interval.

10. The magnetic resonance imaging apparatus according to claim 1, wherein the different blood vessel regions are a large vessel and a non-large vessel in a brain.
